(19) United States
(12) Patent Application Publication
(10) Pub. No.: US 2007/0231833 A1
(43) Pub. Date: Oct. 4, 2007

(54) Labeled antimicrobial peptides and method of using the same to detect microorganisms of interest

(76) Inventors: Steven Michael Arcidiacono, Bellingham, MA (US); Charlene Marie Mello, Rochester, MA (US); Philip E. Pivarnik, Narragansett, RI (US); Andre Senecal, North Smithfield, RI (US)

Correspondence Address: U.S. Army Soldier Systems Center, 15 Kansas Street, Natick, MA, 01760, US

(21) Appl. No.: 11/135,130

(22) Filed: May 23, 2005

(51) Int. Cl. G01N 33/53

(52) U.S. Cl. 435/7.9

(57) ABSTRACT

Labeled antimicrobial peptides and method of using the same to detect a microorganism of interest. In one embodiment, the method involves adding immuno-capture beads to a sample, the immuno-capture beads including capture antibodies coupled to a paramagnetic bead, the capture antibodies being specific for the type of microorganism of interest. After mixing, the target microorganism binds to the capture antibodies. Next, the beads are collected by positioning a magnet close to the sample, and the unbound material is removed from the sample. Then, a solution containing fluorescently-labeled antimicrobial peptide is added to the sample, the labeled peptide binding in great numbers to the immuno-captured microorganism. After removing unbound peptide, the beads are suspended in solution and a magnetic probe is used to collect the beads in a small volume. With the beads thus drawn together, the solution is excited with a laser. Such excitation causes the label to fluoresce, which fluorescence is then detected.

### STATEMENT OF FEDERALLY SPONSORED RESEARCH OR DEVELOPMENT

The invention described herein may be manufactured and used by the U.S. Government for Governmental purposes without the payment of any royalty thereon.

### BACKGROUND OF THE INVENTION

The present invention relates generally to techniques for detecting microorganisms and relates more particularly to a novel technique for detecting microorganisms.

Microorganisms, such as bacteria, viruses, fungi and protozoa, are commonplace in the environment. Although many such microorganisms are innocuous to humans, certain species of microorganisms are pathogenic and pose a serious health risk to people. Exposure to such pathogenic microorganisms may be inadvertent, such as in the case of poorly handled or poorly prepared foods containing Salmonella, Listeria, E. coli O157:H7 or the like, or may be deliberate, such as in the case of biological weapons armed with spores of anthrax or the like. As can readily be appreciated, in view of the above, it is highly desirable to be able to detect the presence of pathogenic microorganisms in various media, such as food, water and air, that are likely to come into human contact. Unfortunately, the presence of pathogenic microorganisms in such media cannot typically be ascertained simply by visual or other sensory examination of the media, but rather, requires the use of specialized testing equipment and procedures. Moreover, because certain pathogenic microorganisms may be lethal in very small doses (for example, in some instances, in doses constituting as few as about ten microorganisms), there is a need for a detection technique that is sensitive enough to detect even very small quantities of such microorganisms.

One type of technique that is commonly used to detect the presence of pathogenic microorganisms in a sample is an antibody sandwich assay, such as an enzyme-linked immunosorbent assay (ELISA). Typically, an ELISA technique uses two types of antibodies, a capture antibody and a detection antibody. The capture antibody has a pair of antigen binding sites and a tail region, the antigen binding sites of the capture antibody being adapted to bind to corresponding antigens present on the pathogen of interest, the tail region of the capture antibody being coupled to a desired substrate, such as a well of a multi-well plate or a magnetic bead. The detection antibody also has a pair of antigen binding sites and a tail region, the antigen binding sites of the detection antibody being adapted to bind to corresponding antigens present on the pathogen of interest, the tail region of the capture antibody being coupled to an enzyme, such as alkaline phosphatase or horseradish peroxidase, each capable of catalyzing colorimetric and chemiluminescent reactions. In this manner, the presence of a microorganism sandwiched between the capture antibody and the detection antibody is indicated by a colorimetric or chemiluminescent reaction resulting from the exposure of an analyte to the enzyme coupled to the detection antibody. Examples of ELISA techniques used in the detection of pathogenic microorganisms may be found in the following U.S. patents, all of which are incorporated herein by reference: U.S. Pat. No. 6,174,667, inventors Huchzermeier et al., which issued Jan. 16, 2001; U.S. Pat. No. 6,124,105, inventors Verschoor et al., which issued Sep. 26, 2000; U.S. Pat. No. 5,294,537, inventor Batt, which issued Mar. 15, 1994; and U.S. Pat. No. 4,486,530, inventors David et al., which issued Dec. 4, 1984.

An alternative technique to the ELISA technique discussed above involves coupling to the detection antibody a fluorescent dye, instead of an enzyme that catalyzes a colorimetric or chemiluminescent reaction.

Unfortunately, there are certain difficulties that are commonly encountered in using the above-described techniques to detect pathogens. First, because of the relatively large size of antibodies (approximately 150,000 Da), it may be difficult in some instances for both a capture antibody and a detection antibody to bind to the same microorganism. Consequently, the sensitivity of the foregoing technique is limited to about 10⁴ bacterial cells/ml. As can readily be appreciated, such sensitivity is not sufficient for real time analysis to ensure the safety of a tested food item. Second, antibodies also suffer from a lack of stability and durability once they are hydrated.

In U.S. Pat. No. 5,750,357, inventors Olstein et al., which issued on May 12, 1998, and which is incorporated herein by reference, there is disclosed a detectable synthetic copolymer that is said to be useful to detect the presence of a microorganism in a test sample. The copolymer comprises repeating monomeric units, which incorporate a population of first monomeric units each comprising a binding agent which binds to a microorganism having multiple binding sites for said binding agent and which further incorporates a population of a second monomeric units each comprising a detectable label or a binding site for a detectable label.

Additionally, in U.S. Pat. No. 6,790,661, inventor Goodnow, which issued on Sep. 14, 2004, and which is incorporated herein by reference, there is disclosed a method for screening for the presence of a clinically relevant amount of bacteria in donor blood or a blood product from a donor mammal, particularly blood or a blood product that will be transferred from the donor mammal to a recipient mammal. The method comprises contacting a sample of the donor blood or a blood product with a set of binding agents that comprises binding agents that specifically bind to Gram-negative bacterial antigen and/or binding agents that specifically bind to Gram-positive bacterial antigen, and determining binding of the set of binding agents to the sample, wherein binding indicates the presence of a clinically relevant amount of Gram-positive bacteria and/or Gram-negative bacteria in the donor blood or blood product and no binding indicates the absence of a clinically relevant amount of Gram-positive bacteria and/or Gram-negative bacteria in the donor blood or blood product. It should be noted that the foregoing method is not specific for particular types of microorganisms, but rather, is directed at broad classes of microorganisms, such as Gram-negative or Gram-positive bacteria.

Moreover, in U.S. Patent Application Publication No. US 2003/0175207, which was published Sep. 18, 2003, and which is incorporated herein by reference, there are disclosed complexes of bacteriocins and metals that are said to be useful in detecting bacteria, particularly Gram-positive bacteria, as well as fungi, and other biological analytes. The complexes are preferably chelated complexes wherein (a) the bacteriocin is a lantibiotic, non-lanthionine containing peptide, large heat labile protein and complex bacteriocin, fusion protein thereof, mixture thereof, and fragment, homolog and variant thereof, and (b) a detectable label comprising a transition or lanthamide metal. The complex preferentially binds to viable Gram-positive or mycobacterial cells. The complex can also bind to Gram-negative bacteria and fungi.

Other documents relating to the detection of microorganisms include the following, all of which are incorporated herein by reference: U.S. Pat. No. 6,630,355, inventors Pivamik et al., which issued Oct. 7, 2003; Liu et al., “Rapid Detection of Escherichia coli O157:H7 Inoculated in Ground Beef, Chicken Carcass, and Lettuce Samples with an Immunomagnetic Chemiluminescence Fiber-Optic Biosensor,” Journal of Food Protection, 66(3):512-7 (2003); DeMarco et al., “Rapid Detection of Escherichia coli O157:H7 in Ground Beef Using a Fiber-Optic Biosensor,” Journal of Food Protection, 62(7):711-6 (1999); Yu et al., “Development of a Magnetic Microplate Chemifluorimmunoassay for Rapid Detection of Bacteria and Toxin in Blood,” Analytical Biochemistry, 261:1-7 (1998); and Zhou et al., “A compact fiber-optic immunosensor for Salmonella based on evanescent wave excitation,” Sensors and Actuators B, 42:169-75 (1997).

### SUMMARY OF THE INVENTION

It is an object of the present invention to provide a new technique for detecting a microorganism of interest present within a sample.

It is another object of the present invention to provide a technique as described above that overcomes at least some of the shortcomings discussed above in connection with existing techniques.

Therefore, according to a first aspect of the invention, there is provided a method for detecting a microorganism of interest present within a sample, said method comprising the steps of (a) providing means for capturing the microorganism of interest, said capturing means comprising a capture antibody having a binding specificity for the microorganism of interest; (b) exposing the sample to the capturing means so as to permit the capture antibody to bind to the microorganism of interest; (c) providing a labeled antimicrobial peptide, said labeled antimicrobial peptide having a binding affinity for the microorganism of interest; (d) exposing any captured microorganism of interest to the labeled antimicrobial peptide so as to permit the antimicrobial peptide to bind to the captured microorganism of interest; and (e) using the labeled antimicrobial peptide to indicate the presence of any captured microorganism of interest.

According to a second aspect of the invention, there is provided a method for detecting a microorganism of interest present within a sample, said method comprising the steps of (a) providing a labeled antimicrobial peptide, said labeled antimicrobial peptide having a non-specific binding affinity for the microorganism of interest; (b) exposing the sample to the labeled antimicrobial peptide so as to permit the labeled antimicrobial peptide to bind to the microorganism of interest; (c) providing means for capturing the microorganism of interest, said capturing means comprising a capture antibody having a binding specificity for the microorganism of interest; (d) exposing any labeled microorganisms to the capture antibody so as to permit the capture antibody to bind to the microorganism of interest; and (e) using the labeled antimicrobial peptide to indicate the presence of any captured microorganism of interest.

According to a third aspect of the invention, there is provided a method for detecting a microorganism of interest present within a sample, said method comprising the steps of (a) providing a labeled antimicrobial peptide, said labeled antimicrobial peptide having a non-specific binding affinity for the microorganism of interest; (b) providing means for capturing the microorganism of interest, said capturing means comprising a capture antibody having a binding specificity for the microorganism of interest; (c) concurrently exposing the sample to both the labeled antimicrobial peptide and the capture antibody so as to permit both the labeled antimicrobial peptide and the capture antibody to bind to the microorganism of interest; and (d) using the labeled antimicrobial peptide to indicate the presence of any captured microorganism of interest.

The present invention is also directed at labeled antimicrobial peptides suitable for use in performing the above-described methods.

Additional objects, as well as features and advantages, of the present invention will be set forth in part in the description which follows, and in part will be obvious from the description or may be learned by practice of the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIG. 1 is a schematic representation of a first embodiment of the method of the present invention;

FIG. 2 is a schematic representation of a second embodiment of the method of the present invention;

FIG. 3 is a schematic representation of a third embodiment of the method of the present invention;

FIG. 4 is a schematic representation of the solution binding assay discussed in Example II;

FIG. 5 is a graph comparing the sensitivity of the labeled antimicrobial peptide Cy5CP1_c to the sensitivity of the labeled antibody Cy5anti-E. coli O157:H7;

FIG. 6 is a graph illustrating the fluorescence detected for various E. coli concentrations by following the procedure discussed in Example III;

FIG. 7 is a graph illustrating the results of the testing discussed in Example IV;

FIG. 8 is a graph illustrating the results of the testing discussed in Example V;

FIG. 9 is a graph illustrating the results of the testing discussed in Example VI;

FIG. 10 is a graph illustrating the binding of various labeled antimicrobial peptides to E. coli 43827, as discussed in Example VII;

FIG. 11 is a graph illustrating the binding of various labeled antimicrobial peptides to S. aureus 27217, as discussed in Example VII;

FIG. 12 is a graph illustrating the binding of various labeled antimicrobial peptides to E. coli O157, as discussed in Example VII; and

FIG. 13 is a chart illustrating the results of the testing discussed in Example VIII.

### DETAILED DESCRIPTION OF PREFERRED EMBODIMENTS

As noted above, the present invention is directed at a new technique for detecting a microorganism of interest present within a sample. According to one aspect of the invention, this technique involves capturing the microorganism of interest using an antibody having a specificity for the microorganism (i.e., a capture antibody) and using a labeled antimicrobial peptide to indicate the presence of the captured microorganism. The microorganism capturing step may be performed before the microorganism labeling step, after the microorganism labeling step, or concurrently with the microorganism labeling step.

The capture antibody used in the microorganism capturing step may be immobilized on a stationary substrate, such as a well of a multi-well plate, a fiber optic or glass waveguide, a membrane or a chromatography column, or may be coupled to a latex bead or a magnetic bead, i.e., for immuno-magnetic separation.

The microorganism labeling step is performed using labeled antimicrobial peptides. Antimicrobial peptides, i.e., naturally-occurring peptides having antimicrobial activity, have received increasing attention over the last several years as a possible means of treating microbial infections. See e.g., U.S. Pat. No. 6,887,981, inventors Zhang et al., which issued May 3, 2005; U.S. Pat. No. 6,872,705, inventor Lyons, which issued Mar. 29, 2005; U.S. Pat. No. 6,809,181, inventors McCray, Jr. et al., which issued Oct. 26, 2004; U.S. Pat. No. 6,790,661, inventor Goodnow, issued Sep. 14, 2004; U.S. Pat. No. 6,713,605, inventors Blecha et al., which issued Mar. 30, 2004; U.S. Pat. No. 6,699,689, inventors Kim et al., which issued Mar. 2, 2004; U.S. Pat. No. 6,541,607, inventor Hansen, which issued Apr. 1, 2003; U.S. Pat. No. 6,482,799, inventors Tuse et al., which issued Nov. 19, 2002; U.S. Pat. No. 6,465,410, inventors Bettiol et al., which issued Oct. 15, 2002; U.S. Pat. No. 6,420,116, inventors Olsen et al., which issued Jul. 16, 2002; U.S. Pat. No. 6,316,594, inventors Kim et al., which issued Nov. 13, 2001; U.S. Pat. No. 6,235,973, inventors Smith et al., which issued May 22, 2001; U.S. Pat. No. 6,183,992, inventors Kim et al., which issued Feb. 6, 2001; U.S. Pat. No. 6,143,498, inventors Olsen et al., which issued Nov. 7, 2000; U.S. Pat. No. 6,042,848, inventors Lawyer et al., which issued Mar. 28, 2000; U.S. Pat. No. 5,936,063, inventors Kim et al., which issued Aug. 10, 1999; U.S. Pat. No. 5,914,248, inventors Kuipers et al., which issued Jun. 22, 1999; U.S. Pat. No. 5,912,230, inventors Oppenheim et al., which issued Jun. 15, 1999; U.S. Pat. No. 5,889,148, inventors Lee et al., which issued Mar. 30, 1999; U.S. Pat. No. 5,885,965, inventors Oppenheim et al., which issued Mar. 23, 1999; U.S. Pat. No. 5,861,275, inventor Hansen, which issued Jan. 19, 1999; U.S. Pat. No. 5,856,127, inventors Powell et al., which issued Jan. 5, 1999; U.S. Pat. No. 5,849,490, inventors Schonwetter et al., which issued Dec. 15, 1998; U.S. Pat. No. 5,844,072, inventors Selsted et al., which issued Dec. 1, 1998; U.S. Pat. No. 5,798,336, inventors Travis et al., which issued Aug. 25, 1998; U.S. Pat. No. 5,750,357, inventors Olstein et al., which issued May 12, 1998; U.S. Pat. No. 5,646,119, inventors Oppenheim et al., which issued Jul. 8, 1997; U.S. Pat. No. 5,631,228, inventors Oppenheim et al., which issued May 20, 1997; U.S. Pat. No. 5,519,115, inventors Mapelli et al., issued May 21, 1996; U.S. Pat. No. 5,447,914, inventors Travis et al., which issued Sep. 5, 1995; Epand et al., “Diversity of antimicrobial peptides and their mechanisms of action,” Biochimica et Biophysica Acta, 1462:11-28 (1999); and Nicolas et al., “Peptides as Weapons Against Microorganisms in the Chemical Defense System of Vertebrates,” Annu. Rev. Microbiol., 49:277-304 (1995), all of which are incorporated herein by reference.

Most antimicrobial peptides are not limited in activity to a specific microorganism, but rather, typically have antimicrobial activity against a rather wide range of microorganisms. The actual mechanism by which antimicrobial peptides function is not, at present, particularly well-understood or critical to the present invention; nevertheless, one of the more common modes of operation appears to be for the antimicrobial peptide to insert itself into the cell membrane of the microorganism in such a way as to create pores therein through which the microbial cytoplasm empties, thereby killing the microorganism. As can readily be appreciated, in order for the antimicrobial peptide to insert itself into the cell membrane of the microorganism, some degree of binding must occur between the antimicrobial peptide and the microorganism. The present invention exploits this binding by using a labeled antimicrobial peptide to bind to a microorganism, thereby indicating the presence of the microorganism.

It should be noted, however, that the antimicrobial peptide of the present invention need not have antimicrobial activity against the target microorganism; rather, all that is required is that the antimicrobial peptide bind to the target microorganism. Many antimicrobial peptides have a binding affinity for large classes of bacteria (e.g., Gram-negative bacteria, Gram-positive bacteria, etc.). In addition, certain antimicrobial peptides may also bind to fungi and viruses as some antimicrobial peptides have been reported to have anti-fungal and antiviral activity.

Because antimicrobial peptides are much smaller than antibodies (about 2000-4000 Da vs. about 150,000 Da) and because antimicrobial peptides tend to bind to cell surfaces via a “blanket” mechanism, the present method has greater sensitivity than do antibody sandwich assays. In addition, because antimicrobial peptides tend to possess a random structure until they interact with a target cell and change to an active conformation, antimicrobial peptides have a robustness and durability not found with antibodies.

Labels that may be coupled to the antimicrobial peptide of the present invention include, but are not limited to, fluorescent tags, such as cyanine 5 dye (Cy5), colorimetric tags, such as alkaline phosphatase, electrochemiluminescent tags, and chemiluminescent tags, such as horseradish peroxidase. Such labels may be covalently bonded directly to the antimicrobial peptide, for example, by an amine group or a sulfhydryl group of the peptide. (Because peptides typically include more amine groups than sulfhydryl groups, bonding to amine groups could permit the peptide to be more highly labeled, thereby increasing sensitivity.) Alternatively, the antimicrobial peptide may be modified, for example, by adding a chemical linker to the peptide, with the label then being covalently bonded to the peptide through the linker. Because of the relatively small size of antimicrobial peptides, as compared to, for example, large reporter molecules, the use of linkers could reduce steric hindrance adversely affecting peptide binding, thereby improving sensitivity.

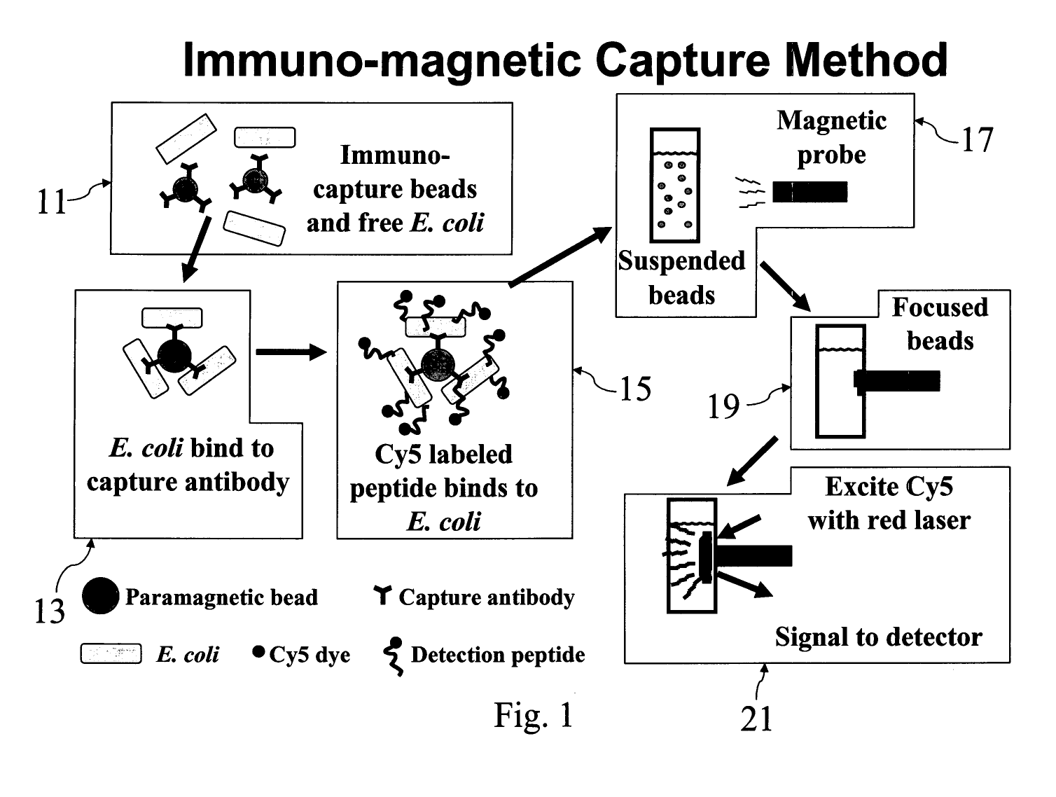

Referring now to FIG. 1, there is schematically shown a first embodiment of a method for detecting the presence of a microorganism of interest in accordance with the teachings of the present invention, said microorganism in the present case, for illustrative purposes only, being E. coli O157:H7. As can be seen at reference numeral 11, a first step of the method involves adding immuno-capture beads to a sample, the immuno-capture beads comprising capture antibodies coupled to a paramagnetic bead. The capture antibodies of the immuno-capture beads are specific for the particular type of microorganism one wishes to detect, which in this case is E. coli O157:H7. Next, as can be seen at reference numeral 13, the immuno-capture beads bind to the E. coli bacteria through the capture antibodies. Next, the immuno-capture beads are collected, typically by positioning a magnet close to the sample, and the unbound materials are removed from the sample, typically by aspiration. Preferably, one or more washing/aspirating cycles are then performed to remove any additional unbound materials from the sample. Then, as can be seen at reference numeral 15, a solution containing labeled antimicrobial peptide (such as a Cy5-labeled antimicrobial peptide) is added to the sample, the labeled antimicrobial peptide binding in great numbers to the immuno-captured bacteria. After removing unbound peptide from the captured bacteria, the beads are then resuspended in solution and a magnetic probe is brought into proximity to the solution, as seen at reference numeral 17. Next, as seen at reference numeral 19, the magnetic probe causes the beads to be drawn together in a small volume. Next, as seen at reference numeral 21, with the beads still drawn together, the sample is excited with a red laser. Such excitation causes the label to fluoresce, which fluorescence is then detected.

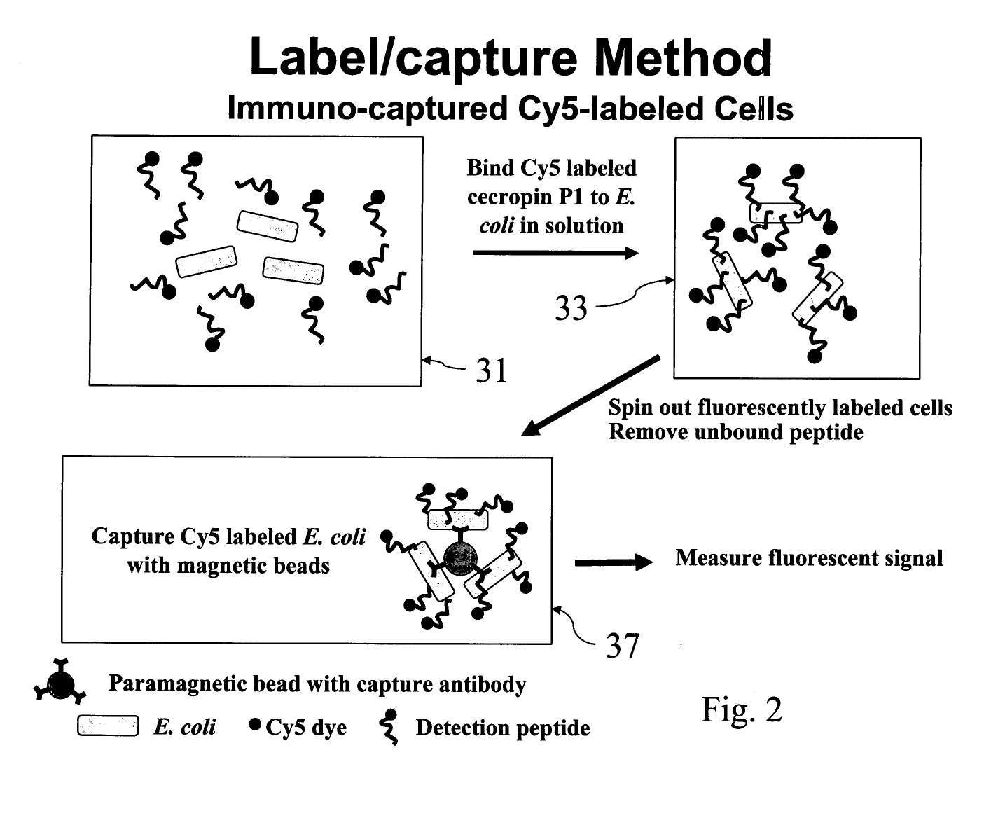

Referring now to FIG. 2, there is schematically shown a second embodiment of a method for detecting the presence of a microorganism of interest in accordance with the teachings of the present invention, said microorganism in the present case, for illustrative purposes only, being E. coli O157:H7. As can be seen at reference numeral 31, a first step of the method involves adding a labeled antimicrobial peptide (such as Cy5-labeled cecropin P1) to a sample. As can be seen at reference numeral 33, the labeled antimicrobial peptide then binds in great numbers to the microorganisms in the sample. (It should be noted that, although only E. coli is shown in FIG. 2, the labeled antimicrobial peptide would likely bind to any other Gram-negative bacteria in the sample as well since Cy5-cecropin P1 has a binding affinity for Gram-negative bacteria.) Next, the sample is subjected to centrifugation, with the pellet including fluorescently-labeled cells and the supernatant including unbound peptide. The supernatant is then removed, and the pellet is put back into solution. Next, as seen at reference numeral 35, immuno-capture beads are added to the solution, the immuno-capture beads comprising capture antibodies coupled to a paramagnetic bead. The capture antibodies of the immuno-capture beads are preferably specific for the particular microorganism of interest, i.e., E. coli O157:H7, so that only that type of microorganism is captured. Next, the immuno-capture beads are collected, typically by positioning a magnet close to the sample, and the unbound materials are removed from the sample, typically by aspiration. Preferably, one or more washing/aspirating cycles are then performed to remove any additional unbound materials from the sample. Next, after removing unbound peptide from the captured bacteria, the captured bacteria are collected using a magnetic probe. The sample is then excited with a laser. Such excitation causes the label to fluoresce, which fluorescence is then detected.

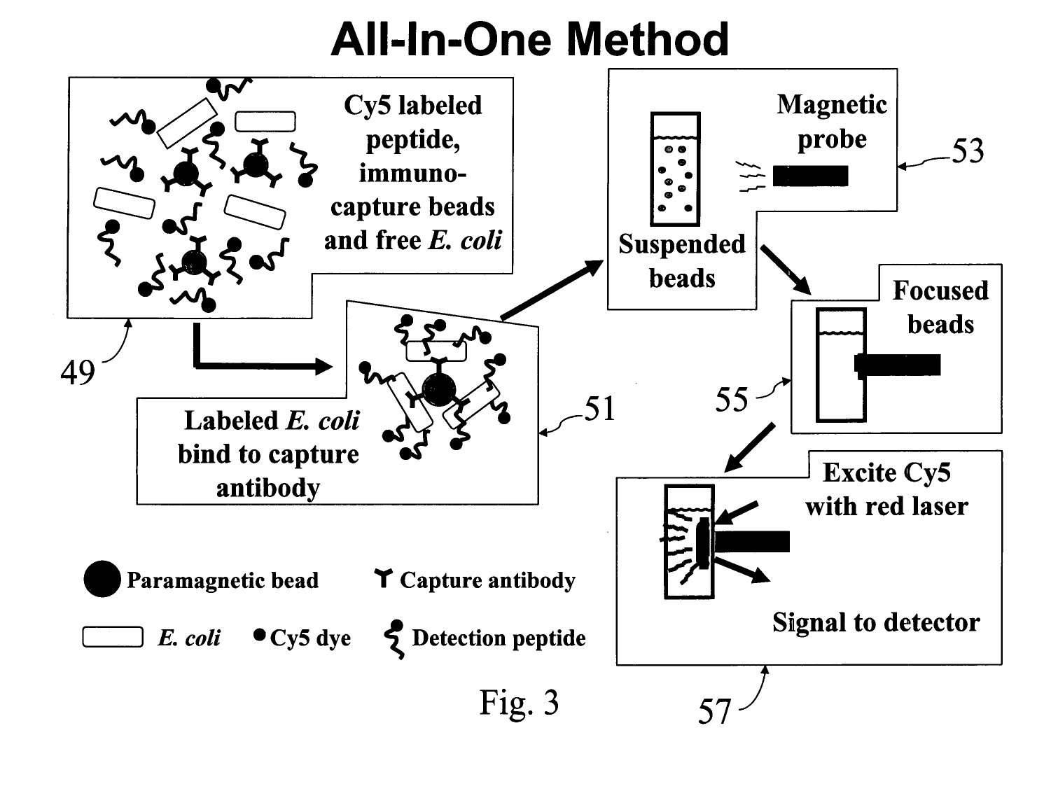

Referring now to FIG. 3, there is schematically shown a third embodiment of a method for detecting the presence of a microorganism of interest in accordance with the teachings of the present invention, said microorganism in the present case, for illustrative purposes only, being E. coli O157:H7. As can be seen at reference numeral 49, a first step of the method involves adding to the solution both a labeled antimicrobial peptide (such as Cy5-labeled cecropin P1) and immuno-capture beads, the immuno-capture beads comprising capture antibodies coupled to a paramagnetic bead. The capture antibodies of the immuno-capture beads are preferably specific for the particular microorganism of interest, i.e., E. coli O157:H7, so that only that type of microorganism is captured. As can be seen at reference numeral 51, soon after being mixed together, the microorganism becomes labeled by the labeled antimicrobial peptide and becomes captured by the immuno-capture beads. (It should be noted that, although only E. coli O157:H7 is shown in FIG. 3, the labeled antimicrobial peptide would likely bind to other Gram-negative bacteria present in the sample as well since Cy5-cecropin P1 has a binding affinity for Gram-negative bacteria.) Next, the immuno-capture beads are collected, typically by positioning a magnet close to the sample, and the unbound materials are removed from the sample, typically by aspiration. Preferably, one or more washing/aspirating cycles are then performed to remove any additional unbound materials from the sample. After removing unbound peptide from the captured bacteria, the beads are then re-suspended in solution and a magnetic probe is brought into proximity to the solution, as seen at reference numeral 53. Next, as seen at reference numeral 55, the magnetic probe causes the beads to be drawn together in a small volume. Next, as seen at reference numeral 57, with the beads still drawn together, the sample is excited with a red laser. Such excitation causes the label to fluoresce, which fluorescence is then detected.

The following examples are provided for illustrative purposes only and are in no way intended to limit the scope of the present invention:

### EXAMPLE I

### Preparation of Cy5-Labeled Antimicrobial Peptides

The antimicrobial peptides cecropin P1 (see Lee et al., “Antibacterial peptides from pig intestine: isolation of a mammalian cecropin,” Proc. Natl. Acad. Sci. U.S.A., 86:9159-62 (1989), which is incorporated herein by reference), PGQ (see Moore et al., “Antimicrobial peptides in the stomach of Xenopus laevis,” J. Biol. Chem., 266:19851-7 (1991), which is incorporated herein by reference), ceratotoxin A (see Marchini et al., “Purification and primary structure of ceratotoxin A and B, two antibacterial peptides from the female reproductive accessory glands of the medfly Ceratitis capitata (Insecta:Diptera),” Insect Biochem. Mol. Biol., 23:591-8 (1993), which is incorporated herein by reference), cecropin A (see Sun et al., “Peptide sequence of an antibiotic cecropin from the vector mosquito, Aedes albopictus,” Biochem. Biophys. Res. Commun., 249:410-5 (1998), which is incorporated herein by reference), CPF3 (see Maloy and Kari, “Structure-activity studies on magainins and other host defense peptides,” Biopolymers (Peptide Science), 37:105-22 (1995), which is incorporated herein by reference), ser5P1, SMAP-29 (see Skerlavaj et al., “SMAP-29: a potent antibacterial and antifungal peptide from sheep leukocytes,” FEBS Letters, 463:58-62 (1999), which is incorporated herein by reference), and pleurocidin (see Cole et al., “Isolation and characterization of pleurocidin, an antimicrobial peptide in the skin secretion of winter flounder,” J. Biol. Chem., 272:12008-13 (1997), which is incorporated herein by reference) and were chemically synthesized by SynPep Corp. (Dublin, Calif.). Each of these peptides was then modified to additionally include a C-terminal cysteine. The resulting sequences were as follows:

Modified Cecropin P1 (CP1_c)

[SEQ ID NO:1]

Ser Trp Leu Ser Lys Thr Ala Lys Lys Leu Glu Asn
1 5 10
Ser Ala Lys Lys Arg Ile Ser Glu Gly Ile Ala Ile
15 20
Ala Ile Gln Gly Gly Pro Arg Cys
25 30
Modified PGQ (PGQ_c)

[SEQ ID NO:2]

Gly Val Leu Ser Asn Val Ile Gly Tyr Leu Lys Lys
1 5 10
Leu Gly Thr Gly Ala Leu Asn Ala Val Leu Lys Gln
15 20
Cys
25
Modified Ceratotoxin A (CTA_c)

[SEQ ID NO:3]

Ser Ile Gly Ser Ala Leu Lys Lys Ala Leu Pro Val
1 5 10
Ala Lys Lys Ile Gly Lys Ile Ala Leu Pro Ile Ala
15 20
Lys Ala Ala Leu Pro Cys
25 30
Modified Cecropin A (CA_c)

[SEQ ID NO:4]

Gly Gly Leu Lys Lys Leu Gly Lys Lys Leu Glu Gly
1 5 10
Val Gly Lys Arg Val Phe Lys Ala Ser Glu Lys Ala
15 20
Leu Pro Val Ala Val Gly Ile Lys Ala Leu Gly Cys
25 30 35
Modified CPF-3 (CPF-3_c)

[SEQ ID NO:5]

Gly Phe Ala Ser Phe Leu Gly Ala Ala Leu Lys Ala
1 5 10
Ala Leu Ile Gly Ala Asn Met Leu Gly Gly Thr Pro
15 20
Gln Gln Cys
25
Modified ser Cecropin P1 (ser5P1_c)

[SEQ ID NO:6]

Ser Trp Leu Ser Ser Lys Thr Ala Lys Lys Leu Glu
1 5 10
Asn Ser Ala Lys Lys Arg Ile Ser Glu Gly Ile Ala
15 20
Ile Ala Ile Gln Gly Gly Pro Arg Cys
25 30
Modified SMAP-29 (SMAP_c)

[SEQ ID NO:7]

Arg Gly Leu Arg Arg Leu Gly Arg Lys Ile Ala His
1 5 10
Gly Val Lys Lys Tyr Gly Pro Thr Val Leu Arg Ile
15 20
Ile Arg Ile Ala Gly Cys
25 30
Modified Pleurocidin (PL_c)

[SEQ ID NO:8]

Gly Trp Gly Ser Phe Phe Lys Lys Ala Ala His Val
1 5 10
Gly Lys His Val Gly Lys Ala Ala Leu His Thr Tyr
15 20
Leu Cys
25

The foregoing peptides were solubilized in phosphate buffered saline (PBS), pH 7.4 at 1 mg/ml and quantitated by BCA Protein Assay Kit (Pierce Biotechnology, Rockford, Ill.). A 3 molar excess of Tris(Carboxyethyl)phosphine (Sigma Chemical Co., St. Louis, Mo.) was added to reduce the peptide. Peptides were labeled at 90 nmol peptide/vial Cy5 dye from Cy5 mono-reactive maleimide kit (Amersham Biosciences, Piscataway, N.J.). Cy5 labeled CP1_c, PGQ_c and SMAP_c were then purified by reverse phase high performance liquid chromatography (RP-HPLC) using a C₄ column, 250×4.6 mm, 5 μm pore size (YMC, Inc., Wilmington, N.C.) using a gradient of acetonitrile in water containing 0.1% trifluoroacetic acid at 1 ml/min flow rate.

| | |
|---|---|
| CP1_c | 0-20%, 5 min. (linear), 20-30%, 20 min. (linear), |
| | 30-95%, 5 min. (linear) |
| PGQ_c | 0-95% acetonitrile, 25 min (linear) |
| SMAP_c | 0-20%, 5 min. (linear), 20-30%, 20 min. (linear), |
| | 30-95%, 5 min. (linear) |

HPLC fractions were lyophilized under vacuum and resuspended in PBS with 0.05% (w/v) Tween 20 (PBST) and analyzed by SDS-PAGE to identify those with labeled peptide. These were then pooled and quantitated by RP-HPLC using unlabeled peptide as a standard curve.

Affinity purified polyclonal antibody to E. coli O157:H7 was obtained from KPL Inc. (Gaithersburg, Md.). 1 mg antibody was fluorescently labeled with Cy5 mono-reactive maleimide kit (Amersham Biosciences) and purified according to manufacturer's instructions.

### EXAMPLE II

### Solution Binding Assay

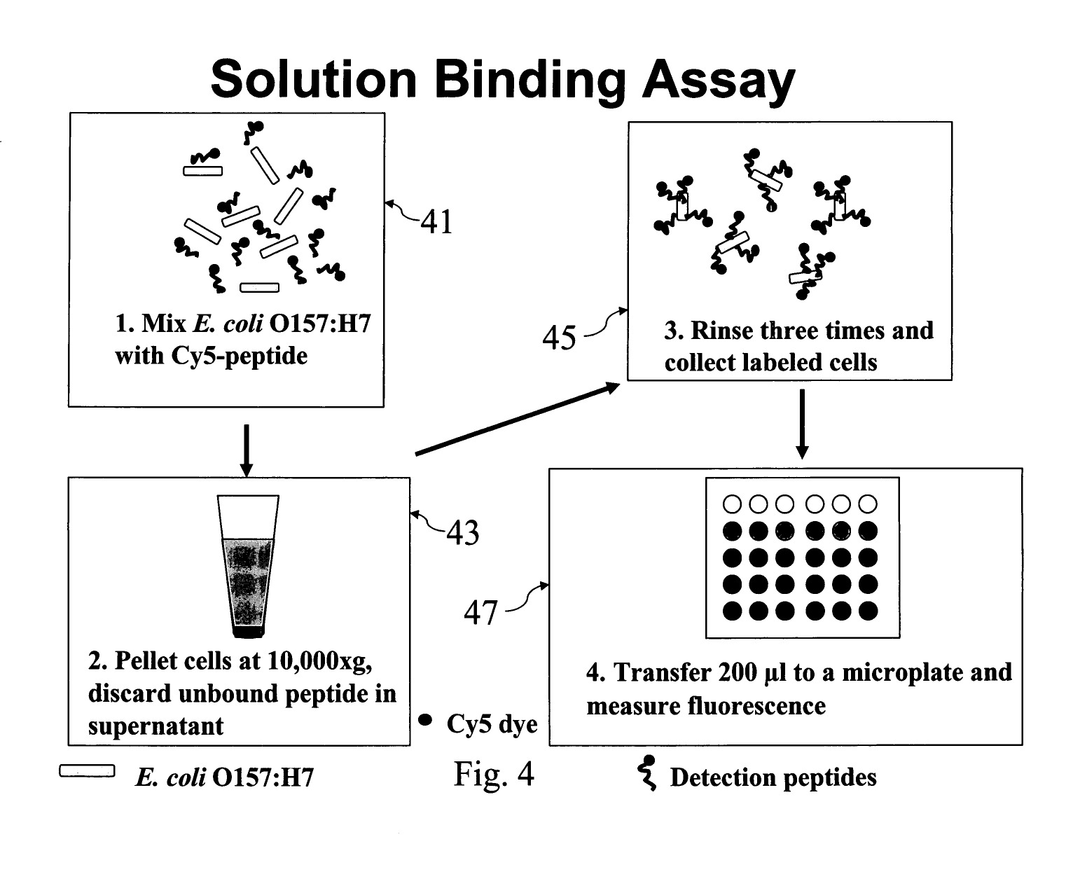

E. coli O157:H7 (ATCC 43888) was grown in Luria broth to OD₆₀₀ 1 (approximately 10⁸ CFU/ml) and washed 2× in equal volume PBST before being resuspended in PBST. 100 μl (10⁷ CFU) cells were added to 900 μl PBST with 5 μg Cy5-CP1_c peptide for 30 minutes at ambient temperature, with rotary mixing (see reference numeral 41 of FIG. 4). Cells were harvested at 10,000×g for 3 minutes (see reference numeral 43 of FIG. 4), the supernatant removed with pipette tip, and washed three times with 1 ml PBST and spun as above (see reference numeral 45 of FIG. 4). Cells were then resuspended in 200 μl PBST and transferred to a black microplate (Nalge Nunc International, Rochester, N.Y.)(see reference numeral 47 of FIG. 4). 900 μl solution containing 5 μg peptide was added to 100 μl buffer without cells and assayed as a negative control. The microplate was imaged using the Storm 860 (Amersham Biosciences, Piscataway, N.J.) using red fluorescence at 1000 V PMT, 200 micron. The image was quantitated by TotalLab version 2003.03 software (Nonlinear Dynamics, Newcastle upon Tyne, UK).

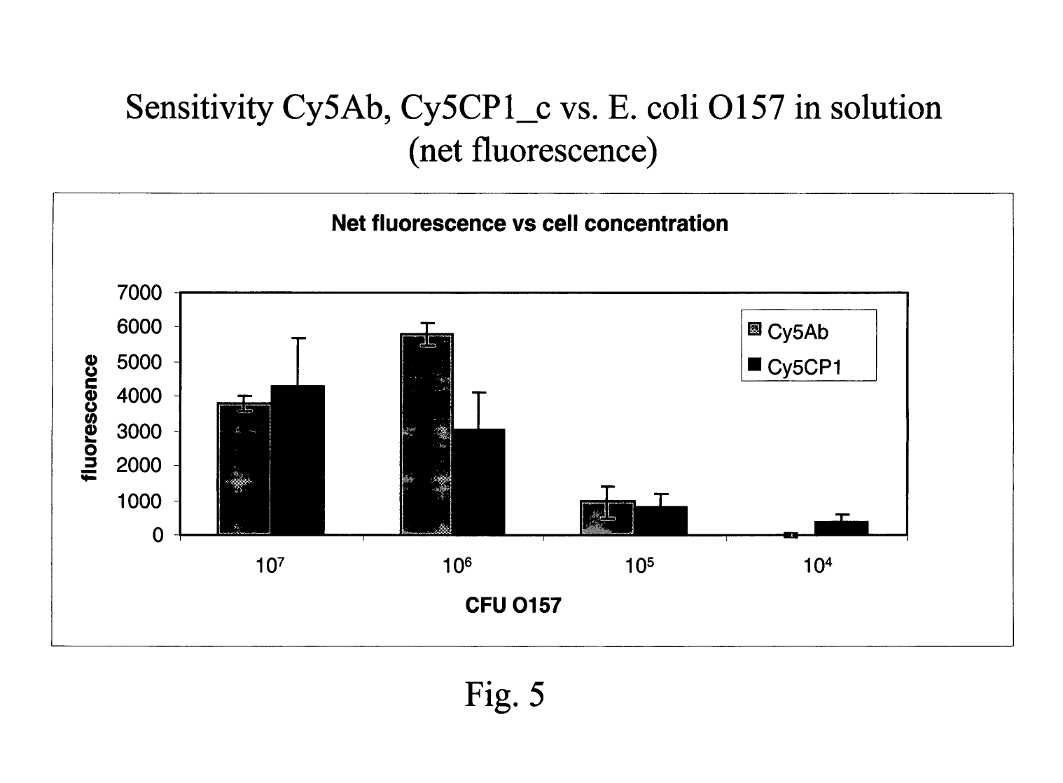

Referring now to FIG. 5, there is shown a graph comparing the sensitivity of the labeled peptide Cy5CP1_c to that of the labeled antibody Cy5CP1_c using the solution binding assay discussed above. As can be seen, the labeled peptide Cy5CP1_c was at least 10 times more sensitive than that of the labeled antibody Cy5anti-O157:H7. (The labeled peptide may be even more sensitive, but 10⁴ CFU was the lowest non-zero concentration tested.)

### EXAMPLE III

### Immuno-Capture of Peptide Labeled Cells

E. coli O157:H7 cells were labeled with Cy5CP1_c in solution as described above. After removal of unbound excess peptide, 1 ml labeled cells was added to 20 μl anti-E. Coli O157:H7 Dyna-beads paramagnetic beads (Dynal Biotech, Browndeer, Wis.) and incubated 30 minutes by rotary mixing. Beads were collected after 2 minutes using a magnetic particle concentrator and washed three times with 1 ml PBST (0.05%). Samples were analyzed on Storm 860 and analyzed by TotaLab software to measure fluorescent signal.

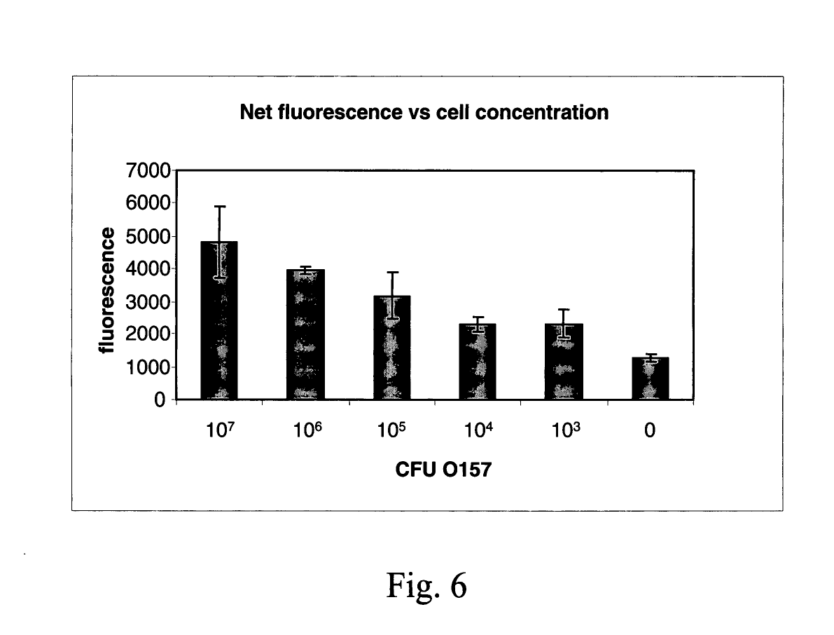

Referring now to FIG. 6, it can be seen that, by following the above procedure, bacteria in concentrations as low as 10³ CFU/ml, the lowest non-zero concentration tested, were capable of being detected.

### EXAMPLE IV

### Solution Binding Assays for Cy5CP1_c and Cy5PGQ_c

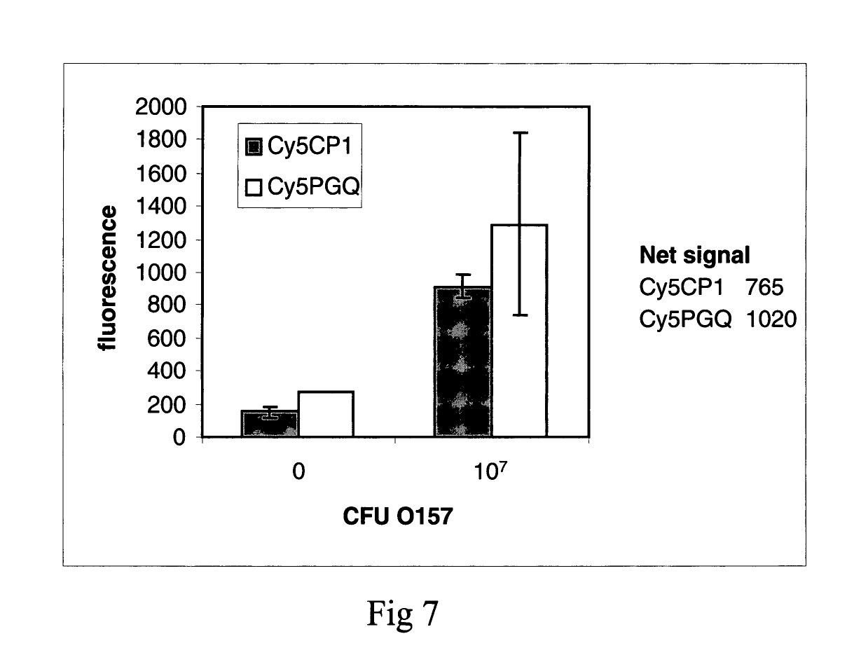

Using the solution binding assay discussed above in Example II, the binding of 2 μg/ml Cy5CP1_c and 2 μg/ml Cy5PGQ_c to E. coli O157:H7 was tested. The results of such testing are shown in FIG. 7. As can be seen, Cy5PGQ_c was capable of binding to E. coli O157:H7 even though it does not have antimicrobial activity against E. coli O157:H7.

### EXAMPLE V

### Labeling of Immuno-Captured Cells

E. coli O157:H7 was prepared in PBST as in Example II. 1 ml E. coli O157:H7 cells were captured with 20 μl anti-E. coli O157 paramagnetic Dyna-beads (Dynal Biotech, Browndeer, Wis.) with rotary mixing for 30 minutes. Beads were collected for 2 minutes using a magnetic particle concentrator and washed three times with 1 ml PBST (0.05%). 1 ml labeled peptide solution at 5 μg/ml in PBST (0.05%) was added to the paramagnetic beads for 30 minutes at ambient temperature with rotary mixing. Beads were collected for 2 minutes using a magnetic particle concentrator and washed three times with 1 ml PBST (0.05%). Beads were re-suspended in 0.5 ml PBST (0.05%) and transferred to 1 ml cuvette. Samples were analyzed on Model FM03 magnetic focusing fluorometer biosensor (Pierson Scientific Associates Inc., Andover, Mass.).

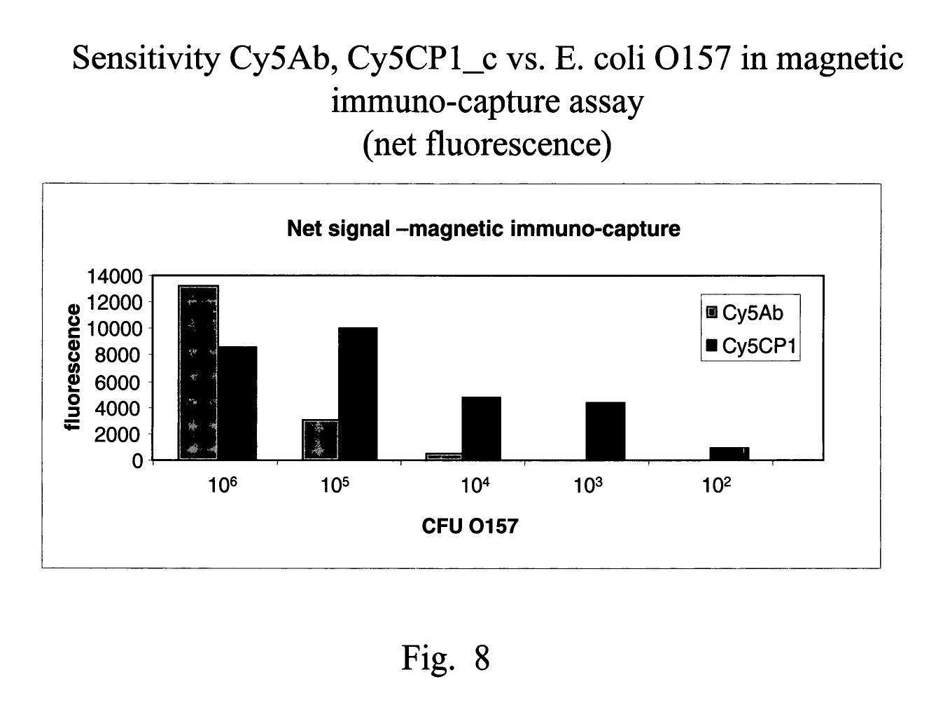

Referring to FIG. 8, it can be seen that a detection sensitivity of 10² CFU/ml was achieved.

### EXAMPLE VI

### All-In-One Assay Method Using Cy5CP1_c

E. coli O157:H7 was prepared in PBST as in Example II. 10⁶ CFU E. coli O157:H7 cells, 20 μl anti-E. coli O157 paramagnetic Dyna-beads (Dynal Biotech, Browndeer, Wis.) and 5 μg/ml Cy5CP1_c in 1 ml were added to a 1.5 ml microfuge tube and rotary mixed for 30 minutes. Beads were collected for 2 minutes using a magnetic particle concentrator and washed three times with 1 ml PBST (0.05%). Beads were re-suspended in 0.5 ml PBST (0.05%) and transferred to 1 ml cuvette. Samples were analyzed on Model FM03 magnetic focusing fluorometer biosensor. This All-In-One method was compared to immuno-capture assay. Samples were analyzed on Model FM03 magnetic focusing fluorometer biosensor (Pierson Scientific Associates Inc., Andover, Mass.). Detection sensitivity of 10² CFU was achieved.

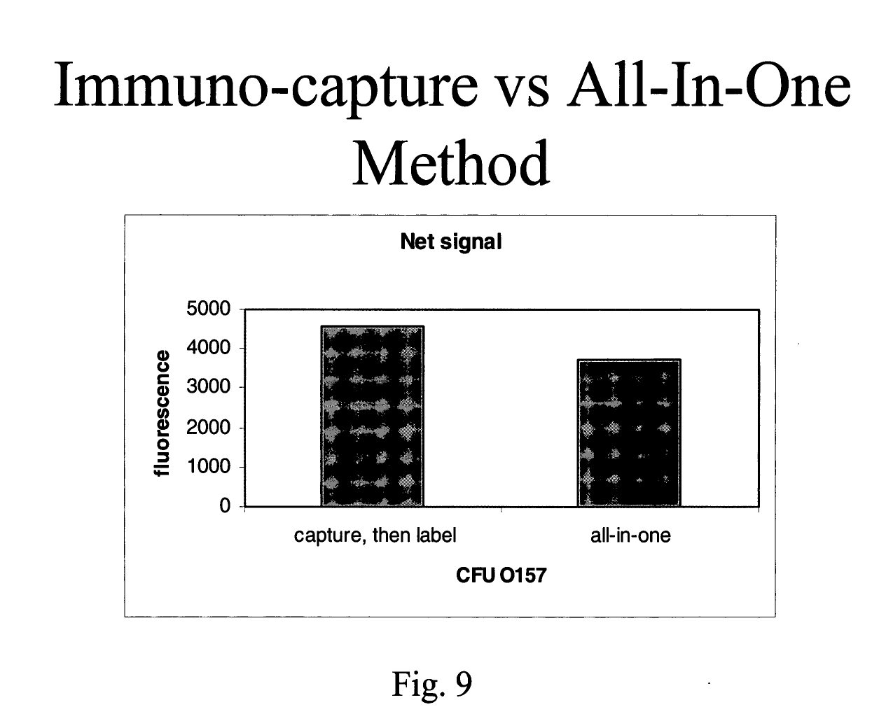

Referring to FIG. 9, it can be seen that the All-In-One method gave similar signal in one-half the time as the immuno-capture assay.

### EXAMPLE VII

### Screening Cy5 Labeled Peptides by Solution Binding Assay

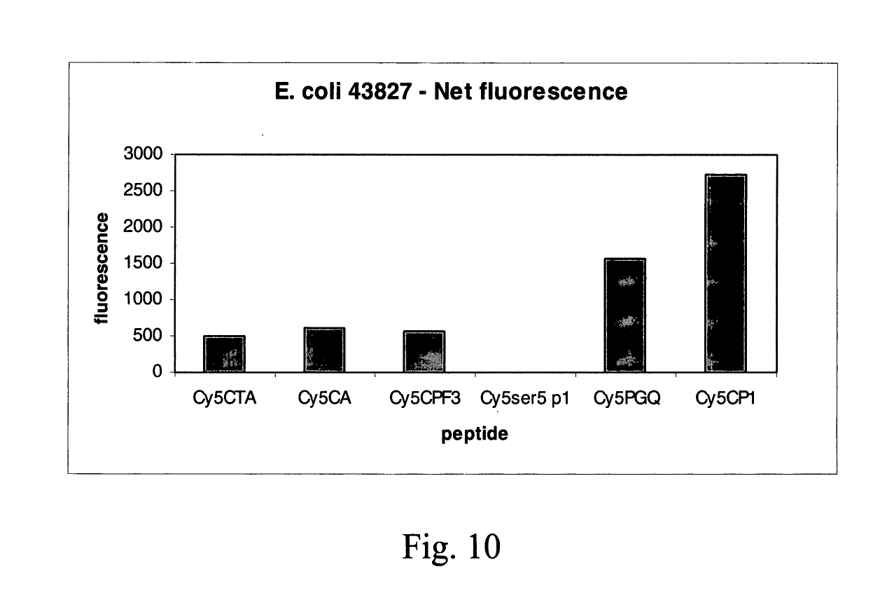

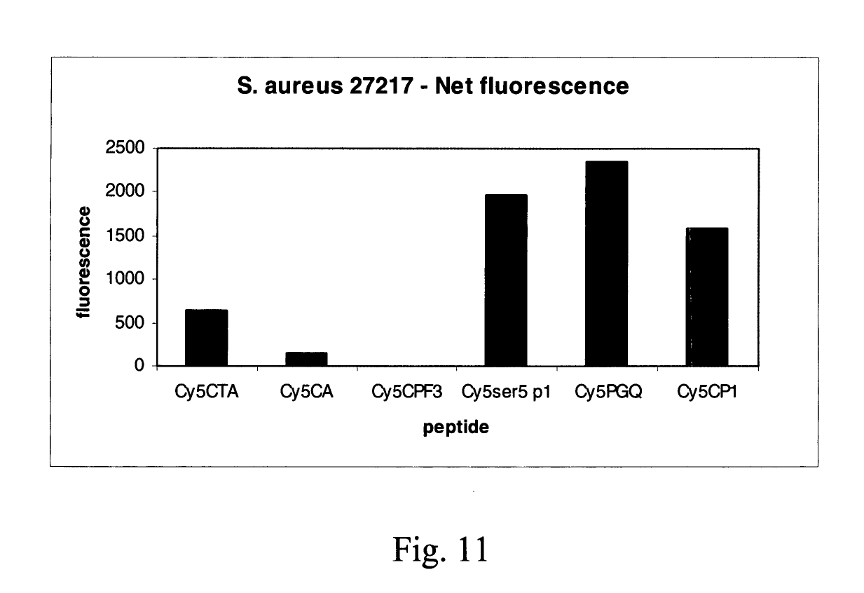

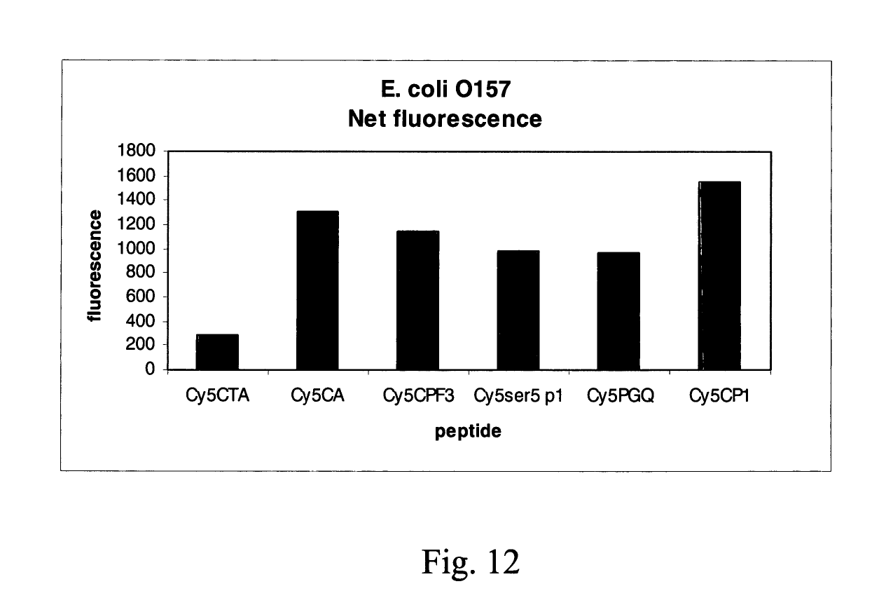

Using the solution binding assay discussed in Example II, various peptides from the labeling reactions of Example I were diluted in PBST (0.05%) to 5 μg/ml and tested against 10⁶ CFU/ml cells. 5 μg HPLC purified Cy5CP1 (see Example I) was run as a control. The samples were transferred to black microtiter plate for Storm 860 imaging and TotaLab analysis. Referring to FIGS. 10, 11 and 12, the ability of these peptides to bind to different microorganisms is demonstrated.

### EXAMPLE VIII

### Detection Sensitivity of HPLC Purified Cy5 Labeled Peptides

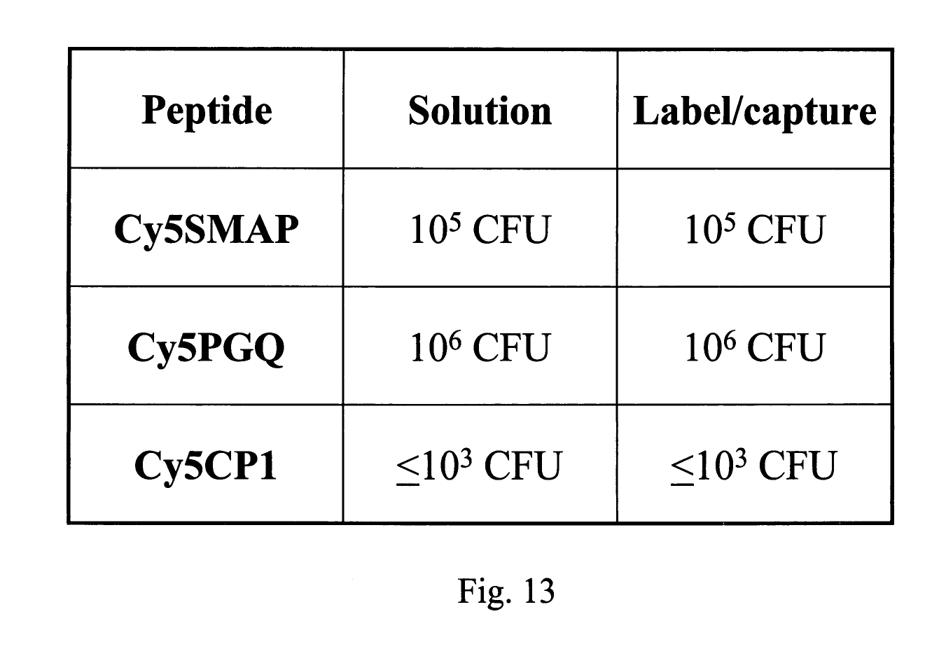

Using the solution binding assay discussed in Example II and the label/capture method discussed in Example III, the binding of 5 μg/ml HPLC purified Cy5CP1_c, Cy5SMAP_c, and Cy5PGQ_c was tested against dilutions of E. coli O157 to determine detection sensitivity. The results of such testing are shown in FIG. 13. As can be seen, Cy5CP1_c is, by far, the most sensitive of the labeled antimicrobial peptides tested for E. coli O157 detection.

The embodiments of the present invention recited herein are intended to be merely exemplary and those skilled in the art will be able to make numerous variations and modifications to it without departing from the spirit of the present invention. All such variations and modifications are intended to be within the scope of the present invention as defined by the claims appended hereto.

What is claimed is:

1. A method for detecting a microorganism of interest present within a sample, said method comprising the steps of:
(a) providing means for capturing the microorganism of interest, said capturing means comprising a capture antibody having a binding specificity for the microorganism of interest;
(b) exposing the sample to the capturing means so as to permit the capture antibody to bind to the microorganism of interest;
(c) providing a labeled antimicrobial peptide, said labeled antimicrobial peptide having a binding affinity for the microorganism of interest;
(d) exposing any captured microorganism of interest to the labeled antimicrobial peptide so as to permit the antimicrobial peptide to bind to the captured microorganism of interest; and
(e) using the labeled antimicrobial peptide to indicate the presence of any captured microorganism of interest.

2. The method as claimed in claim 1 wherein said capturing means further comprises a paramagnetic bead, said capture antibody being coupled to said paramagnetic bead.

3. The method as claimed in claim 1 wherein said capturing means further comprises an immobilized substrate, said capture antibody being coupled to said immobilized substrate.

4. The method as claimed in claim 3 wherein said immobilized substrate is selected from the group consisting of a well of a multi-well plate, a fiber optic waveguide, a glass waveguide, a membrane and a chromatography column.

5. The method as claimed in claim 1 wherein said capture antibody has a binding specificity for E. coli O157:H7.

6. The method as claimed in claim 1 wherein said labeled antimicrobial peptide comprises a detectable tag coupled to an antimicrobial peptide, said detectable tag being selected from the group consisting of a fluorescent tag, a colorimetric tag, an electrochemiluminescent tag and a chemiluminescent tag.

7. The method as claimed in claim 6 wherein said detectable tag is covalently bonded to the antimicrobial peptide through a C-terminal cysteine residue added to the antimicrobial peptide.

8. The method as claimed in claim 6 wherein said fluorescent tag is cyanine 5 dye (Cy5).

9. The method as claimed in claim 6 wherein said labeled antimicrobial peptide is selected from the group consisting of Cy5CP1_c, Cy5PGQ_c, Cy5CTA_c, Cy5CA_c, Cy5CPF-3_c, Cy5ser5P1_c, Cy5SMAP_c, and Cy5PL_c.

10. The method as claimed in claim 9 wherein said labeled antimicrobial peptide is Cy5CP1_c.

11. A method for detecting a microorganism of interest present within a sample, said method comprising the steps of:
(a) providing a labeled antimicrobial peptide, said labeled antimicrobial peptide having a non-specific binding affinity for the microorganism of interest;
(b) exposing the sample to the labeled antimicrobial peptide so as to permit the labeled antimicrobial peptide to bind to the microorganism of interest;
(c) providing means for capturing the microorganism of interest, said capturing means comprising a capture antibody having a binding specificity for the microorganism of interest;
(d) exposing any labeled microorganisms to the capture antibody so as to permit the capture antibody to bind to the microorganism of interest; and
(e) using the labeled antimicrobial peptide to indicate the presence of any captured microorganism of interest.

12. The method as claimed in claim 11 wherein said capturing means further comprises a paramagnetic bead, said capture antibody being coupled to said paramagnetic bead.

13. The method as claimed in claim 11 wherein said capture antibody has a binding specificity for E. coli O157:H7.

14. The method as claimed in claim 11 wherein said labeled antimicrobial peptide comprises a detectable tag coupled to an antimicrobial peptide, said detectable tag being selected from the group consisting of a fluorescent tag, a calorimetric tag, an electrochemiluminescent tag and a chemiluminescent tag.

15. The method as claimed in claim 14 wherein said detectable tag is covalently bonded to the antimicrobial peptide through a C-terminal cysteine residue added to the antimicrobial peptide.

16. The method as claimed in claim 14 wherein said fluorescent tag is cyanine 5 dye (Cy5).

17. The method as claimed in claim 16 wherein said labeled antimicrobial peptide is selected from the group consisting of Cy5CP1_c, Cy5PGQ_c, Cy5CTA_c, Cy5CA_c, Cy5CPF-3_c, Cy5ser5P1_c, Cy5SMAP_c, and Cy5PL_c.

18. The method as claimed in claim 16 wherein said labeled antimicrobial peptide is Cy5CP1_c.

19. A method for detecting a microorganism of interest present within a sample, said method comprising the steps of:
(a) providing a labeled antimicrobial peptide, said labeled antimicrobial peptide having a non-specific binding affinity for the microorganism of interest;
(b) providing means for capturing the microorganism of interest, said capturing means comprising a capture antibody having a binding specificity for the microorganism of interest;
(c) concurrently exposing the sample to both the labeled antimicrobial peptide and the capture antibody so as to permit both the labeled antimicrobial peptide and the capture antibody to bind to the microorganism of interest; and
(d) using the labeled antimicrobial peptide to indicate the presence of any captured microorganism of interest.

20. The method as claimed in claim 19 wherein said capturing means further comprises a paramagnetic bead, said capture antibody being coupled to said paramagnetic bead.

21. The method as claimed in claim 19 wherein said labeled antimicrobial peptide comprises a detectable tag coupled to an antimicrobial peptide, said detectable tag being selected from the group consisting of a fluorescent tag, a colorimetric tag, an electrochemiluminescent tag and a chemiluminescent tag.

22. The method as claimed in claim 21 wherein said detectable tag is covalently bonded to the antimicrobial peptide through a C-terminal cysteine residue added to the antimicrobial peptide.

23. The method as claimed in claim 19 wherein the microorganism of interest is E. coli and wherein said labeled antimicrobial peptide is selected from the group consisting of Cy5CP1_c, Cy5PGQ_c, Cy5CTA_c, Cy5CA_c, Cy5CPF-3_c, Cy5ser5P1_c, Cy5SMAP_c, and Cy5PL_c.

24. A labeled antimicrobial peptide selected from the group consisting of Cy5CP1_c, Cy5PGQ_c, Cy5CTA_c, Cy5CA_c, Cy5CPF-3_c, Cy5ser5P1_c, Cy5SMAP_c, and Cy5PL_c.


SEQUENCE LISTING (8 sequences)

SEQ ID NO: 1 - protein, 32 aa, Artificial Sequence
  C-terminal cysteine added to cecropin P1
SWLSKTAKKLENSAKKRISEGIAIAIQGGPRC

SEQ ID NO: 2 - protein, 25 aa, Artificial Sequence
  C-terminal cysteine added to PGQ
GVLSNVIGYLKKLGTGALNAVLKQC

SEQ ID NO: 3 - protein, 30 aa, Artificial Sequence
  C-terminal cysteine added to ceratotoxin A
SIGSALKKALPVAKKIGKIALPIAKAALPC

SEQ ID NO: 4 - protein, 36 aa, Artificial Sequence
  C-terminal cysteine added to cecropin A
GGLKKLGKKLEGVGKRVFKASEKALPVAVGIKALGC

SEQ ID NO: 5 - protein, 27 aa, Artificial Sequence
  C-terminal cysteine added to CPF-3
GFASFLGAALKAALIGANMLGGTPQQC

SEQ ID NO: 6 - protein, 33 aa, Artificial Sequence
  C-terminal cysteine added to ser cecropin P1
SWLSSKTAKKLENSAKKRISEGIAIAIQGGPRC

SEQ ID NO: 7 - protein, 30 aa, Artificial Sequence
  C-terminal cysteine added to SMAP-29
RGLRRLGRKIAHGVKKYGPTVLRIIRIAGC

SEQ ID NO: 8 - protein, 26 aa, Artificial Sequence
  C-terminal cysteine added to pleurocidin
GWGSFFKKAAHVGKHVGKAALHTYLC